(19) United States
(12) Patent Application Publication
(10) Pub. No.: US 2025/0333783 A1
(43) Pub. Date: Oct. 30, 2025

(54) METHOD OF CHARACTERIZING CAPPED RNA

(71) Applicant: Vestlandets Innovasjonsselskap AS, Bergen (NO)

(72) Inventors: Eivind Dala Valen, Bergen (NO); Adnan Muhammad Niazi, Godvik (NO); Jan Inge Øvrebø, Bergen (NO)

(21) Appl. No.: 18/871,434

(22) PCT Filed: Jun. 8, 2023
(86) PCT No.: PCT/EP2023/065421

§ 371 Date: Dec. 3, 2024

(30) Foreign Application Priority Data
Jun. 9, 2022 (NO) 20220658

(51) Int. Cl. C12Q 1/6869; C12Q 1/6883; C12Q 1/6886

(52) U.S. Cl. CPC C12Q 1/6869; C12Q 1/6883; C12Q 1/6886; C12Q 2600/158

(57) ABSTRACT

The present invention provides a method of characterizing a capped ribonucleic acid (RNA) using sequencing, wherein the capped RNA is a ribonucleic acid (RNA) with its native 5′ cap, the method comprising the steps of: (i) oxidation of the vicinal diol of the native 5′ cap of the capped RNA; (ii) ligation of a polynucleotide adapter via a linker to the oxidized diol of the native 5′ cap providing an extended polynucleotide construct and (iii) sequencing at least a portion of the extended polynucleotide construct, wherein said portion includes the native cap. The present invention further provides a method of identifying whether a genetic marker specific for a condition is present in a sample which utilises the method of the invention; as well as kits for use in the methods of the invention. The invention further provides a method of characterising an RNA with a native 5′ cap, which method comprises sequencing at least a portion of a polynucleotide construct comprising said capped RNA and a polynucleotide adapter ligated via a linker to said cap, wherein the linking moiety is formed from the vicinal diol of the native 5′ cap, and wherein said portion includes the native 5′ cap.

### FIELD OF THE INVENTION

The invention relates to the field of characterizing 5′ capped ribonucleic acids.

### BACKGROUND OF THE INVENTION

Ribonucleic acid or RNA is a central component of all life. In eukaryotes, RNA polymerases transcribe DNA in the cell's nucleus to RNA. From their creation to their degradation, these RNAs are under extensive regulation. This regulation is directed not only by their nucleotide sequence and their promoter but also by post- and co-transcriptional modifications attached to the RNA. One of the most ubiquitous of these modifications in eukaryotes is the 5′ cap.

Several types of RNAs receive 5′ caps including messenger RNAs (mRNA). mRNA caps are 5′-terminal modifications that are typically attached co-transcriptionally on RNA Polymerase II (pol-II) transcribed transcripts in eukaryotes. When the transcribed pre-mRNA is 20-30 nt long, a series of enzymatic reactions add an inverted methylated guanosine (m⁷G) to the 5′-end of the nascent RNA with a 5′-5′ triphosphate bridge. This terminal inverted m⁷G base is called Cap0 (also represented by the notation m⁷GpppN₁pN₂p-RNA, where N₁ and N₂ are the first and the second transcribed nucleotide of the pre-mRNA, respectively, and p represents the phosphate group(s) between them).

In addition to the terminal m⁷G, the RNA caps may contain other modifications, the most common of which is a 2′-O methylation or Nm modification of the first and/or second transcribed base. This methylation can lead to different caps depending on which nucleotide receives this modification, e.g., cap1 (m⁷GpppN₁mpN₂p-RNA), cap2(m⁷GpppN₁mpN₂mp-RNA), or cap2-1(m⁷GpppN₁pN₂mp-RNA). Some organisms also have methylation on the third and fourth transcribed bases resulting in larger and potentially more complex cap structures.

Additionally, when the first transcribed nucleotide (N1) is an adenine (A) base, it can have a methylation at the N6 position. This methylation can then result in cap0-m⁶A (m⁷Gpppm⁶ApN₂p-RNA), cap1-m⁶Am (m⁷Gpppm⁶AmpN₂p-RNA), cap2-m⁶Am (m⁷Gpppm⁶AmpN₂mp-RNA), and cap2-1-m⁶A (m⁷Gpppm⁶ApN₂mp-RNA) cap structures (Cowling 2019). Moreover, on some RNAs, the terminal m⁷G in cap0 can be further methylated by to form a trimethylguanosine (TMG)/m₃^{(2,2,7)}G cap structure. All the caps discussed so far have a terminal m⁷G and are collectively known as canonical caps.

Recently, a non-canonical (NC) class of caps has been discovered in eukaryotes. These caps have a metabolite effector instead of the terminal m⁷G. Unlike m⁷G caps, which are added during transcription, some of the non-canonical caps can initiate transcription by serving as a non-canonical initiation nucleotide (NCIN). Two of the most well-known NC caps are the NAD+ and NADH caps formed using the oxidized and reduced forms of nicotinamide adenine dinucleotide (NAD), respectively. Other NC caps include the flavin adenine dinucleotide (FAD) caps, uridine diphosphate glucose (UDP-Glc), and uridine diphosphate N-acetylglucosamine (UDP-GlcNAc) (Hu, Flynn, and Chen 2021). Many more non-canonical caps such as those containing the different variations dinucleoside polyphosphates (NpₙNs) have been found to exist in bacteria (Hudeček et al. 2020).

In Nanopore sequencing, the RNA is fed through a protein nanopore suspended in a membrane that separates two ionic buffer-filled wells. A voltage applied across the membrane sends a current through the pore that a translocating RNA strand can disrupt. Any modifications on the RNA, including cap methylations, can result in a distinct signature in the pore current, which can, theoretically, be decoded to predict the type of modification.

To sequence the RNA through a Nanopore, a DNA adapter containing a motor protein is attached to the 3′-end of the of the RNA. This motor protein feeds the RNA through the nanopore in the 3′-to-5′ direction at a slow and controlled speed. If there is no motor protein to control the translocation of the RNA, the RNA, under the influence of applied voltage, passes through the pore at such a fast pace that it is not be possible to acquire enough current measurements per base to properly decode the translocated bases during basecalling. The motor protein, therefore, ensures that each translocating base spends a sufficient amount of time in the pore so that enough current measurements can be recorded for accurate basecalling later on.

When the ratcheting motor protein reaches the 5′-end of the RNA however, it loses its grip on the RNA and falls off from the RNA strand. Consequently, approximately 10-20 terminal nucleotides at the 5′ end of the RNA pass through the pore at such a fast speed that the current signal for these terminal bases cannot be acquired reliably. As a result, the 5′-ends including the RNA caps cannot be characterized using the default Nanopore sequencing protocols.

In many sequencing methods and biological experiments, RNA cannot be used directly but first must be copied into complementary cDNA (cDNA) which is then subsequently used. To copy RNA into cDNA, reverse transcriptase is used which reads the RNA from 3′ to 5′ and makes a DNA strand complementary to the RNA. However, most reverse transcriptases fall off from the RNA before completely reaching the 5′-end thereby yielding 5′-truncated cDNAs. The cap-jumping method (Efimov et al. 2001; Merenkova and Edwards 2000) extends the 5′-end of the RNA by ligating an adapter oligonucleotide extension to the native 5′-cap. This 5′ oligonucleotide extension enables the reverse transcriptase to go over the entire RNA and make a cDNA that contains the complete copy of the original RNA. This method was developed so that the start of the transcript could be identified through sequencing cDNA with standard DNA sequencing approaches. It yielded, however, little information about the nature of the 5′ cap other than what the first transcribed bases are.

WO 2019/226822 A1 (Mulroney et al. 2021) discloses a method of analysing capped ribonucleic acids using nanopore sequencing which involves the ligation of an adapter polynucleotide to the 5′ cap of an RNA molecule. The adapter polynucleotide serves to maintain contact with a motor protein while the 5′ end of the RNA transcript traverses the nanopore. However, Mulroney et al. 2021 provides very little information as to how the adapter polynucleotide is attached to the capped RNAs, saying only that the attachment depends on the type of 5′ cap that is present, and that it may be facilitated by polymerase-mediated extension or enzyme-mediated ligation. The Examples given in Mulroney et al. 2021 provide no teaching as to how to produce a capped RNA molecule with the adapter polynucleotide attached, as is required for their method. It is therefore concluded that Mulroney et al. 2021 does not disclose how to carry out the method of their invention in a manner that is sufficiently clear for the skilled person to reproduce.

Due to the deficiencies in the detail provided in Mulroney et al. 2021, the present inventors were unable to elucidate how to carry out the method of the disclosure. However, they were able to resolve the method once they found a related thesis (Mulroney, 2020) which contains very similar, if not identical, figures/results to those disclosed in Mulroney et al. 2021. While Mulroney et al. 2021 suggests that the adapter polynucleotide can be added to the 5′ cap of the RNA molecule using any available enzyme, Mulroney, 2020 makes clear that the addition of the adapter polynucleotide is done in a multi-step reaction, where two specific enzymes are mentioned. First, the native 5′ cap from RNA is decapped using yDcpS enzymes. This enzyme can decap certain RNAs (for an exhaustive list of yDcpS-compatible caps list, see (Wulf et al. 2019)) by severing the pyrophosphate bond between gamma and beta phosphates in the triphosphate bridge of the native 5′ (canonical) cap. yDcpS can cleave off the m7G moiety of Cap0, Cap1 and Cap2 caps in this way. The decapped RNAs with diphosphate ends are then recapped with a non-native cap (3′-(O-Propargyl)-GTP) using Vaccinia capping enzyme (VCE). This non-native cap makes the recapped RNA amenable to ligation via Copper-catalysed click chemistry to an oligonucleotide adapter carrying an azide moiety at its 3′-end. The adapted molecules comprising the oligonucleotide adapter, the non-native cap, and the RNA transcript are then sequenced.

Furthermore, the vaccinia capping enzyme used in prior art approach can only recap RNAs with diphosphate ends. Currently there are no known decapping enzymes that can decap caps such as NADH, NAD⁺, FAD, etc. in such a way that leaves the diphosphate group behind on the residual RNA chain. Using the yDcpS enzyme as disclosed in Mulroney 2020, this diphosphate can only be generated for RNAs that have a Cap0, Cap1 or Cap2 native cap.

yDcspS cannot cleave off TMG caps. A purely hypothetical enzyme would be needed to cleave TMG caps in order to leave the diphosphate required for VCE-mediated non-native cap ligation. Similarly, there is no known enzyme that can cleave off an NAD cap in the required position to leave the diphosphate necessary for VCE-mediated ligation. The skilled person could consider that NudC could be used instead to cleave off the NAD cap, but this does not leave a suitable diphosphate as required for VCE-mediated ligation of the non-native cap. A purely hypothetical enzyme would be needed to cleave the nicotinamide moiety of the NAD cap without also cleaving the beta phosphate.

If these hypothetical enzymes were known and used to carry out the Mulroney, 2020 method, the resultant non-native capped RNAs would look the same, regardless of whether the RNA originally had a Cap0, TMG or NAD cap. For example, an RNA sample might contain two populations of RNA, one carrying m⁷G caps and the other carrying TMG caps. By following prior art approach, the decapping step would remove the terminal m⁷G, and m3^{(2,2,7)}G moieties in the RNA molecules, leaving behind diphosphate ends. Recapping with 3′-(O-Propargyl)-GTP and oligo ligation would yield RNA molecules all having the same non-native cap. The difference of cap types between the two different capped RNA populations is lost because the m⁷G and m3^{(2,2,7)}G moieties which are the distinguishing features of these caps had to be removed for prior art ligation approach to work. Therefore, important native cap information is completely lost using the method of Mulroney, 2020. Only modifications on the transcribed bases could in principle be distinguished from each other using the method of Mulroney, 2020.

In summary, although Mulroney et al. 2021 in combination with Mulroney, 2020 allows for ligating an adapter to the 5′-end of recapped RNAs, thereby enabling the sequencing of the full-length RNA transcripts with a cap, the native 5′ cap moiety is sacrificed in the process as it is replaced by a non-native cap. Consequently, the signal obtained for the cap from the nanopore belongs not to the native RNA cap, but to a non-native cap which was substituted for the native cap. Owing to these shortcomings, the approach of WO 2019/226822 (Mulroney et al. 2021) will fail to sequence or distinguish between all the different caps that might be present in a biological sample.

Other existing methods for determining native cap structures require severing off the cap from their respective transcripts and then using either chromatography- or mass-spectrometry-based methods to separate and identify the different cap types. These bulk methods lack transcript-level specificity—or even gene-level specificity, for that matter—and can, at the most, only give a relative abundance estimate of different cap structures present in an RNA sample. The lack of methods for cap structure prediction at single-molecule resolution represents a significant bottleneck in understanding the transcriptome-wide role of different cap structures. A single-molecule cap prediction method can shed light on the factors that influence the presence of one or the other cap type on a transcript and inform about the role that these different caps play in the fate of their respective transcripts.

The object of the present invention was to provide a method for characterizing a capped RNA at a single molecule level. Put in other words, the object of the invention was to provide methods for analyzing both the native 5′ cap together with the RNA in one assay.

### BRIEF SUMMARY OF THE INVENTION

The inventors surprisingly found tools to analyze both the native 5′ cap together with the RNA in one assay by providing a method as indicated in the claims. In particular, the present invention relates to a method of characterizing a capped RNA using sequencing, wherein the capped RNA is a ribonucleic acid (RNA) with its native 5′ cap, the method comprising the steps of:
i) oxidation of the vicinal diol of the native 5′ cap of the capped RNA;
ii) ligation of a polynucleotide adapter via a linker to the oxidized diol of the native 5′ cap providing an extended polynucleotide construct and
iii) sequencing at least a portion of the construct including the native cap of the extended polynucleotide construct.

Alternatively written, the present invention relates to a method of characterising a capped ribonucleic acid (RNA) using sequencing, wherein the capped RNA is a ribonucleic acid (RNA) with it native 5′ cap, the method comprising the steps of:
i) oxidation of the vicinal diol of the native 5′ cap of the capped RNA;
ii) ligation of a polynucleotide adapter via a linker to the oxidized diol of the native 5′ cap providing an extended polynucleotide construct and
iii) sequencing at least a portion of the extended polynucleotide construct, wherein said portion includes the native cap.

In one preferred embodiment, the method comprises a further step of reductive amination of the oxidized vicinal diol formed in i) between steps i) and ii). In one further preferred embodiment, the method comprises following further steps between steps ii) and iii): nucleotide material precipitation, RNA purification, poly-A removal and/or poly-A tailing. In one embodiment the RNA purification may be bead RNA purification.

The sequencing may be carried out e.g., with nanopore sequencing.

Preferably, the linker comprises amine groups (NH₂). These allow bonding between sequencing adapter (OTE) and RNA cap dialdehyde groups (resulting from oxidation of vicinal diols). Preferably, the linker is an ethylenediamine. More preferably, the linker should possess structural features allowing passage of the cap and the linker, through a nanopore, in such a way that the ionic current signal allows successful identification of cap type. In another preferred embodiment, the linker is a bond.

In one preferred embodiment of the invention, the polynucleotide adapter comprises an introduced amino group, preferably at its 3′ end.

In one preferred embodiment of the invention, the polynucleotide adapter may be ligated to the 3′ end of the RNA in addition to the 5′ end, or to both 5′ and 3′ ends of the RNA. In this embodiment, a sequencing motor protein may be attached to the polynucleotide adapter that is ligated to the 5′ end of the RNA or directly to the native 5′ cap. In a further preferred embodiment, ligating the polynucleotide adapter to the 5′ end enables sequencing of the RNA in the 3′ to the 5′ direction on a nanopore sequencer. Put in other words, according to one preferred embodiment of the present invention, ligating the polynucleotide adapter at the 5′-cap enables nanopore sequencing in the 3′ to the 5′ direction. In a further embodiment, ligating the polynucleotide adapter to the 5′ end enables sequencing of the RNA in the 5′ to the 3′ direction on a nanopore sequencer. Put in other words, according to one embodiment of the present invention, ligating the polynucleotide adapter at the 5′-cap enables nanopore sequencing in the 5′ to the 3′ direction.

Furthermore, the extended RNA construct can be reverse transcribed into full length double-stranded cDNA that can be sequenced for the characterization of the 5′ ends of the RNA. The sequencing may be carried out by any sequencing platform that can use cDNA as input including but not limiting to Nanopore, Illumina or Pacific Biosciences sequencing. In an additional preferred embodiment, steps i) and ii) of the method of the present invention can be used in the creation of full-length cDNA from RNA.

According to the present invention the extended RNA construct may be characterized by ionic current signature produced during its translocation through the nanopore.

Preferably according to the method, a plurality of native 5′ caps present in the polynucleotide construct sample may be sequenced using a single assay. That is to say a plurality of native 5′ caps present in a sample of polynucleotide constructs may be sequenced using a single assay. Thus a sample comprising capped RNAs of interest, wherein the structure of the caps are not known, may be analysed in a single assay. The sample is treated and each capped RNA undergoes the same sequence of chemical reactions, regardless of the individual cap structures.

Preferably, the native 5′ cap contains a vicinal diol, preferably a periodate-susceptible vicinal diol. In this case, the periodate may react with the diol. The diol relating to the present invention may be a 2′,3′; 1′,2′; or a 3′,4′ diol.

Preferably, the native 5′ cap is selected from the group consisting of tri-methylated m₃^{2,2,7}G, m⁷G, G, NADH, NAD⁺, FAD, Glc-UDP, GlcNAc-UDP and NpₙN. Thus, the native 5′ cap may be canonical or non-canonical.

According to the present invention the polynucleotide adapter may be RNA or DNA. Examples of polynucleotide adapters are depicted in SEQ ID NO.: 1 and SEQ ID NO.: 2.

In a further aspect, the invention provides a method of characterising an RNA with a native 5′ cap, in which method comprises sequencing at least a portion of a polynucleotide construct comprising said capped RNA and a polynucleotide adapter ligated via a linker to said cap through a morpholine ring (formed from the vicinal diol of the native 5′ cap), wherein said portion includes the native 5′ cap.

In a further aspect the present invention provides a method of characterising an RNA with a native 5′ cap, which method comprises sequencing at least a portion of a polynucleotide construct comprising said capped RNA and a polynucleotide adapter ligated via a linker to said cap, wherein the linking moiety is formed from the vicinal diol of the native 5′ cap, and wherein said portion includes the native 5′ cap. For example, the polynucleotide adapter may be ligated via a linker to said cap through a morpholine ring.

Preferred features of the adapter, linker and cap are as described herein in relation to other aspects of the invention. The morpholine ring is the structure that is formed by the preferred method of oxidation and ligation described herein. In this embodiment, the sequencing step may therefore be separated temporally or spatially from the steps of generating the extended polynucleotide construct.

In a further aspect, sequencing information generated in iii) is inputted into a classifier of sequence information in order to characterise said capped RNA, preferably the sequencing information generated in iii) is inputted into a classifier of sequence information in order to identify the native 5′ cap of said capped RNA. Example 6 herein describes how a classifier may be generated and used in the performance of the methods of the invention.

In a further aspect, the sequencing step (iii) includes determination of the native cap structure.

A further aspect of the invention relates to a method of identifying whether a genetic marker specific for a condition is present in a sample wherein the method comprises the steps:
i) obtaining an mRNA sample of a subject
ii) carrying out the sequencing method of the invention
iii) analyzing one or more genetic markers specific for the condition
iv) comparing the sequence of the mRNA sample with a control sample and
v) evaluating the results.

Evaluation of the results may be used for a possible diagnosis.

Cap-methylation plays an important role in sensing of “self-RNA”, where non-capped transcripts will trigger RNA degradation through the activation of the innate immune response (Schuberth-Wagner et al. 2015, Devarkar, Wang, and Miller, 2016). Misregulation of the capping process can therefore have deleterious effect on gene expression. In addition, some oncogenes, such as PI3K, stimulate oncogenic growth through cap-dependent translation (Dunn et. al. 2019, Bjornsti et. al. 2004). This invention can thus be utilized to identify capping status of downstream targets of oncogenic signaling pathways. Further, this method can be developed for the purpose of diagnostics of diseases or conditions driven by genetic markers with a specific cap-status.

For discovery of disease-related genetic markers (or genetic markers relating to specific healthy conditions), the profile/map of caps and their respective transcripts are compared across disease and healthy samples (or across subject and control samples in case of healthy conditions) to identify differentially capped RNA transcripts. These transcripts are then subjected to further investigation.

The condition may be a cancer, viral disease, bacterial disease, or an autoimmune disease. The method can be used as a part of a diagnosis. The method is carried out in vitro.

For discovery of disease-related genetic markers (or genetic markers relating to specific healthy conditions), the profile/map of caps and their respective transcripts are compared across disease and healthy samples (or across subject and control samples in case of healthy conditions) to identify differentially capped RNA transcripts.

Further use of this technique could be expanded to any biological sample where sequencing of cap types would be of interest. This could include pathogens, environmental sequencing, geo sequencing, animal or plant samples.

In a further aspect, the invention relates to a kit for characterizing a ribonucleic acid with its native 5′ cap comprising the reagents for the method of the present invention and instructions for carrying out the method.

In one further aspect, the invention relates to a non-transitory computer readable medium comprising instructions for method of characterizing the native 5′ capped RNA of the present invention.

In another aspect, the invention relates to a computing device comprising a processor and the above-mentioned non-transitory computer readable medium, preferably wherein the computing device is part of a system comprising a nanopore sequencing device.

### BRIEF DESCRIPTION OF THE DRAWINGS

FIG. 1: Examples of canonical cap structures as shown also in Table 1. Periodate-susceptible vicinal diols (hydroxyl groups) at the native 5′-cap are highlighted in filled black rectangles. These vicinal diols are targeted for ligation of a polynucleotide adapter to the native 5′-cap in the present invention.

FIG. 2: Examples of non-canonical cap structures. Periodate-susceptible vicinal diols (hydroxyl group) in these native 5′-caps are highlighted in filled black rectangles. These vicinal diols are targeted for ligation of a polynucleotide adapter to the native 5′-cap in the current invention.

FIG. 3: Structure of the sequenced RNA construct comprising:
A) polynucleotide adapter (oligonucleotide extension (OTE));
B) linker;
C) native 5′ cap (canonical/non-canonical);
D) mRNA chain;
E) poly-A tail; and
F) ONT adapter

FIG. 4: The method of the present invention (Cap-jumping protocol) using a linker. The method of the invention ligates the polynucleotide adapter, i.e. oligonucleotide extension adapter (OTE), using a dedicated/separate linker molecule.
A) An OTE is treated with sodium periodate to oxidize vicinal diols at the 3′ end. The product of the reaction is then reacted with ethylene diamine (or other diamines) linker molecule in the presence of sodium cyanoborohydride. A reductive amination reaction takes place that joins the linker to the 3′ end of the OTE; a morpholine ring is created in the process.
B) The native capped RNA is treated with sodium periodate to oxidize vicinal diols at both the 5′ cap and the 3′-most nucleotide.
C) Products of reactions in A and B are combined in the presence of sodium cyanoborohydride. This causes the previously prepared OTE to ligate with both the 5′ end and 3′ of the capped RNA. The OTE at the 3′ end is ligated in reverse orientation.
D) The reverse-oriented OTE at the 3′-end of the capped RNA is removed by oligo(dT)-guided RNase H cleavage of the poly-A tail. Subsequent poly-A tailing is followed by the standard library preparation protocol for Nanopore sequencing.

FIG. 5: Signal obtained for an m7G-capped (A) and NAD-capped (B) RNA sequenced using method of FIG. 4. Different parts of the signal are labelled with a letter and represent: a. Signal for the ONT DNA adapter; b. Signal for the poly-A tail; c. Signal for the RNA chain; d. Signal for the native m7G cap, the linker chemistry, and a portion of the oligonucleotide extension.

FIG. 6: The method of the present invention (Cap-jumping protocol), wherein a separate linker molecule is not used because the synthetic oligonucleotide extension adapter (OTE) contains an amine moiety at its 3′-end.
A) The native capped RNA is first treated with sodium periodate to oxidize vicinal diols at both the 5′ cap and 3′-most nucleotide.
B) The oxidized capped RNA from step A is reacted with a synthetic oligonucleotide extension OTE with 3AmMC6T amino modifier at the 3′ end. The reaction is carried out in the presence of sodium cyanoborohydride and results in the ligation of the OTE to both the 5′ end and 3′ of the capped RNA. The OTE ligated at the 3′-end of the RNA is in the reverse orientation.
C) The reverse-oriented OTE at the 3′-end of the capped RNA is removed by oligo(dT)-guided RNaseH cleavage of the poly-A tail. Subsequent poly-A tailing is followed by the standard library preparation protocol for Nanopore sequencing.

FIG. 7: Signal obtained for an m7G-capped RNA sequenced using method of FIG. 6. Different parts of the signal are labelled with letter and represent: a. Signal for the ONT DNA adapter; b. Signal for the poly-A tail; c. Signal for the RNA chain; d. Signal for the native m7G cap, the linker chemistry, and a portion of the oligonucleotide extension.

### DETAILED DESCRIPTION OF THE INVENTION

The present invention relates to a method of characterizing a capped RNA using sequencing, wherein the capped RNA is a ribonucleic acid (RNA) with its native 5′ cap, the method comprising the steps of:
i) oxidation of the vicinal diol of the native 5′ cap of the capped RNA;
ii) ligation of a polynucleotide adapter via a linker to the oxidized diol of the native 5′ cap providing an extended polynucleotide construct and
iii) sequencing at least a portion of the construct including the native cap of the extended polynucleotide construct.

Alternatively written, the present invention relates to a method of characterising a capped ribonucleic acid (RNA) using sequencing, wherein the capped RNA is a ribonucleic acid (RNA) with it native 5′ cap, the method comprising the steps of:
i) oxidation of the vicinal diol of the native 5′ cap of the capped RNA;
ii) ligation of a polynucleotide adapter via a linker to the oxidized diol of the native 5′ cap providing an extended polynucleotide construct and
iii) sequencing at least a portion of the extended polynucleotide construct, wherein said portion includes the native cap.

The inventors provide an approach that ligates the polynucleotide adapter to the vicinal diols of the native 5′-cap itself. Consequently, it is the native cap that gets sequenced e.g. on the Nanopore and different native caps will generate different current signatures that can be used to decode them. Furthermore, with the method of the invention it is possible to ligate the polynucleotide adapter to a vast majority of both canonical and non-canonical caps that have vicinal diols in them using a single assay. Examples of caps with periodate-susceptible vicinal diols include the trimethylated, monomethylated and unmethylated G-caps, and NADH, NAD⁺, FAD, Glc-UDP, GlcNAc-UDP, and NpₙN caps.

The method of the invention covalently attaches an oligonucleotide to the 5′-cap itself. However, since the terminal m7G cap is inverted and is connected to the rest of the transcript with an unusual 5′-5′ bond it was impossible to enzymatically ligate a polynucleotide adapter, i.e., an oligonucleotide extension (OTE), to the m7G cap itself, using commercially available ligases. According to the present invention one can, however, chemically link the OTE to the ribose sugar backbone of the terminal m7G nucleotide instead. This can extend the 5′-end of the transcripts without removing the protective m7G cap. With the m7G cap intact, 5′-end degradation can be avoided, and hence more reliable cap-type predictions can be obtained. The method is named cap jumping because many reverse transcriptases can go across (or ‘jump’) the cap and reverse transcribe the covalently bonded oligonucleotide.

To ligate polynucleotide adapter (OTE) to the cap, the cap jumping approach exploits an important feature of every capped transcript: Each canonical capped mRNA transcript has a 2′,3′ diol present at both of its extreme ends—5′ and 3′.

If this terminal 2′,3′-cis diol can be made amenable for linking, then an oligonucleotide can be bound to the 5′-end without removing the protective m7G cap. Towards this end, the capped RNA is first treated with sodium periodate NaIO₄ which oxidizes the 2′,3′-cis diols at both ends of the RNA and coverts them dialdehydes. This reaction makes the RNA amenable for linking an OTE to both 5′ caps and 3′ ends.

A similar NaIO₄ treatment is carried out for the OTE which has a phosphate group at the 5′ end and a 2′,3′-cis diol at the 3′ end. Hence the oxidation of the diol happens only at the 3′-end of the oligonucleotide. Next ethylene diamine dichloride NH₂CH₂CH₂NH₂·2HCl is added in the presence of a reducing agent such as sodium cyanoborohydride NaCNBH₃. This results in a reductive amination of the dialdehyde that causes one of the nitrogen atoms of ethylene diamine dichloride to click into the 3′-end dialdehyde of the polynucleotide adapter, OTE to form a morpholine ring.

To ligate the 3′-modified OTE and previously prepared mRNA transcripts, they are added together in the presence of sodium cyanoborohydride, NaCNBH₃. This causes another round reductive amination reaction in which one of the available nitrogen atoms from the 3′ end of OTE clicks into di-aldehydes present at both 3′ and 5′ ends of the RNA. However, since the OTE may be ligated to both 3′ and 5′ of mRNA and the OTE at the 3′ end is in reverse orientation (3′-5′). The reverse-ligated OTE at the 3′-end needs to be cleaved off because its reverse orientation makes it impossible to ligate the nanopore adapter to it that carries the motor protein. Towards this end, an oligo(dT) is added in the presence of RNase H which cleaves off the mRNA at the poly(A) tail thereby removing the 3′-oligonucleotide in the process.

In this way, an OTE can extend the 5′-end. However, in doing so two morpholine rings—separated by a CH₂CH₂ carbon chain—are sandwiched between the transcript and the OTE, and the endogenous poly(A) tail is also lost.

With this approach, surprisingly it is possible to sequence not only canonical caps, but most non-canonical caps as well because these caps have an exposed diol that can be targeted by the periodate treatment. It is furthermore surprising that the extended polynucleotide constructs generated in accordance with the methods of the invention are able to pass through a nanopore and be sequenced, the ligated adaptors may not have traversed the pore and/or they may not have interacted appropriately with the motor protein.

The sequencing runs may be performed on a nanopore sequencing device. Examples of sequencing devices are Flongle or a MinION, e.g. using the SQK-RNA002 kit.

### Definitions

It is to be noted that the term “a” or “an” entity refers to one or more of that entity; for example, “preparation”, is understood to represent one or more preparations. As such, the terms “a” (or “an”), “one or more”, and “at least one” can be used interchangeably herein.

The term “capped RNA or polynucleotide” relates to an RNA or DNA (e.g. cDNA) that has a cap attached to the RNA or polynucleotide chain. The term “native 5′ cap” refers to the original cap or indigenous cap produced by the organism. Thus the native 5′ cap will generally be a naturally occurring cap as described herein and in particular it does not include the synthetic cap 3-(O-propargyl)-GTP or the synthetic cap 3′-azido-ddGTP.

The term “canonical or non-canonical cap” means two different types of caps. The caps that have a terminal m⁷G are collectively referred to as canonical caps. Also, a non-canonical (NC) class of caps has been discovered in eukaryotes. These caps can have a metabolite effector instead of the terminal m⁷G. Unlike m⁷G caps, which are added during transcription, many of the non-canonical caps can initiate the transcription by serving as a non-canonical initiation nucleotide (NCIN). Moreover, the non-canonical cap is added by the RNA polymerase itself in contrast to the canonical caps in which the m⁷G cap is added by the capping complex. Two of the most well-known NC caps are the NAD⁺ and NADH caps formed using the oxidized and reduced forms of nicotinamide adenine dinucleotide (NAD), respectively. Other NC caps include the flavin adenine dinucleotide (FAD) caps, uridine diphosphate glucose (UDP-Glc), and uridine diphosphate N-acetylglucosamine (UDP-GlcNAc) (Hu, Flynn, and Chen 2021). Many more non-canonical caps such as those containing the different variations dinucleoside polyphosphates (NpₙNs) have been found to exist in bacteria (Hudeček et al. 2020).

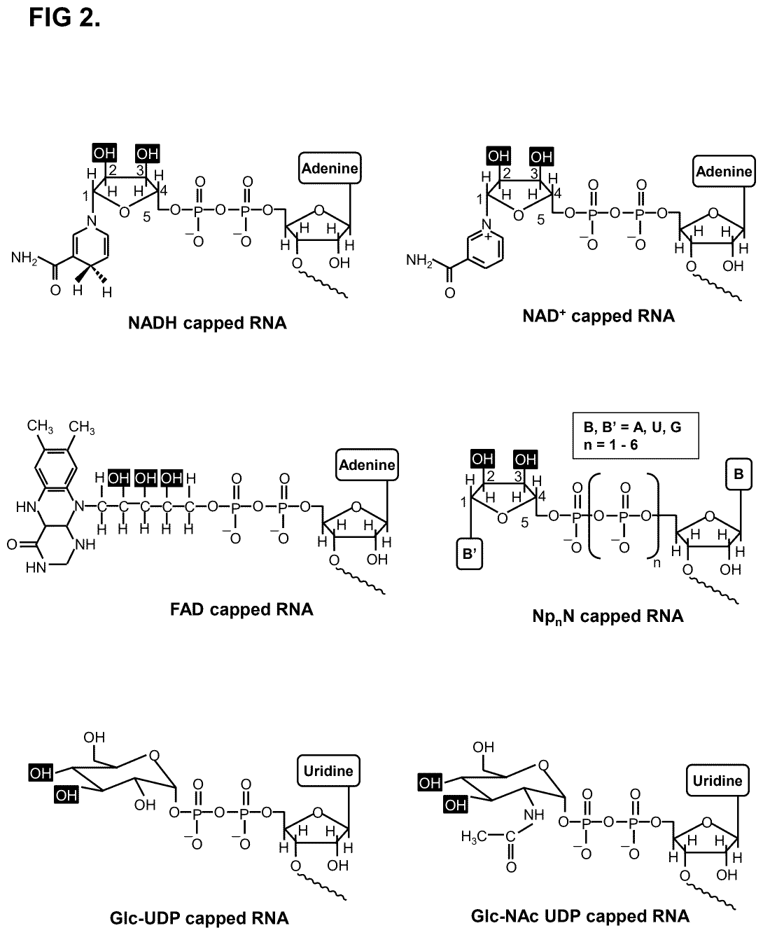

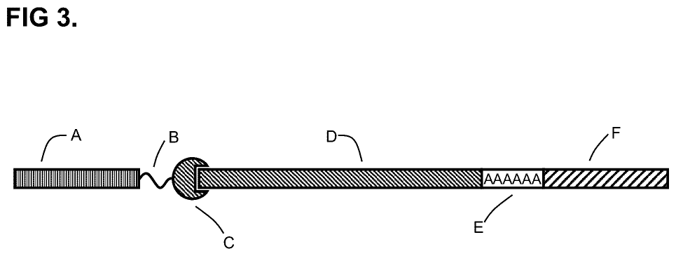

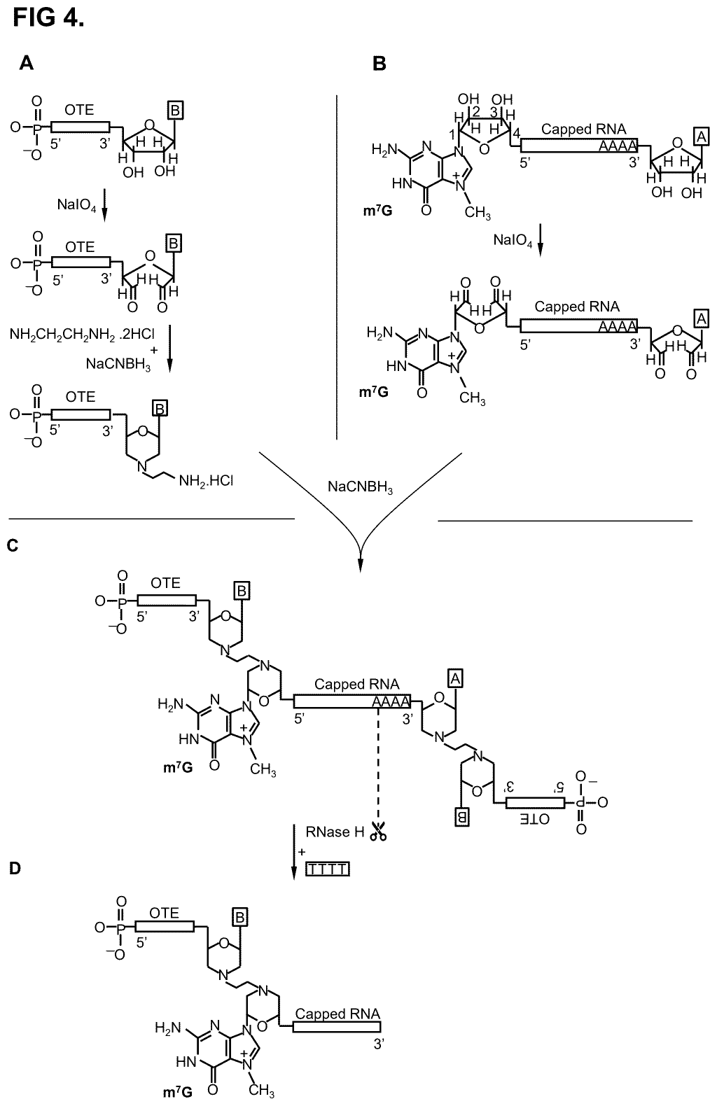

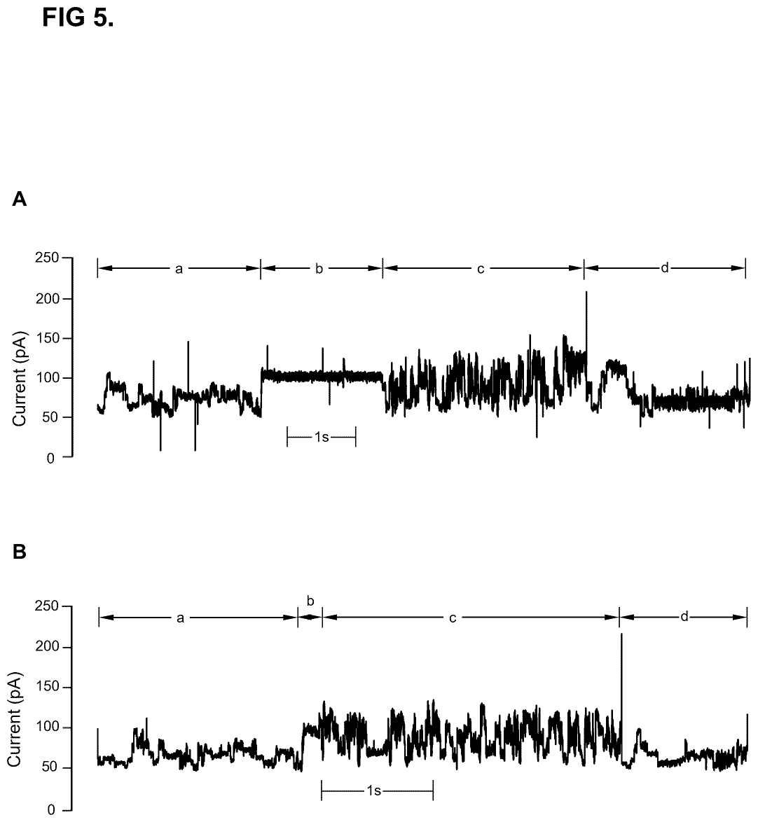

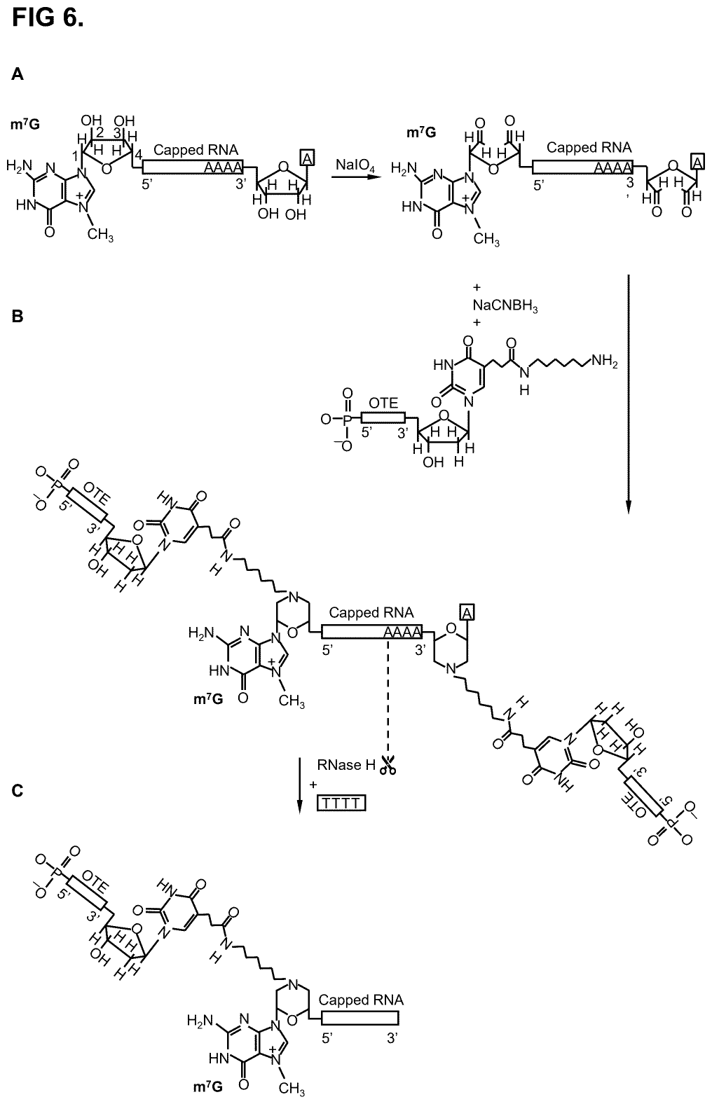

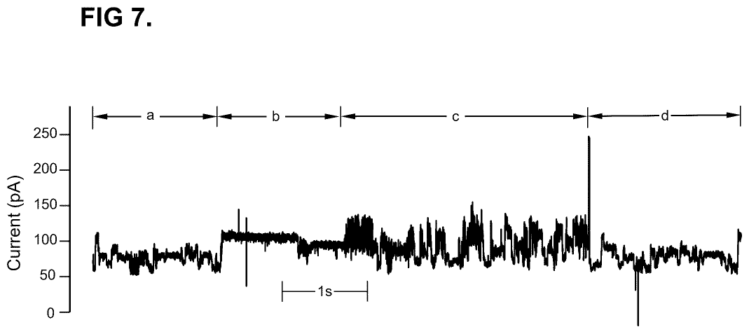

In the following different canonical structures are indicated; see also FIG. 2 and Table 1 below.

### Cap0

The m⁷G of the minimal cap structure cap0, plays a major role in various processes that happen during the life-cycle of mRNA. In the nucleus, the cap-binding complex binds to the m⁷G to prevent degradation of the RNA and to facilitate the export of the mRNA from the nucleus to cytoplasm (Lewis and Izaurralde 1997). In the cytoplasm, the translation initiation factor elF4E binds to m⁷G and helps in the circularization of RNA into a loop for efficient translation of the mRNA into protein by ribosomes (Preiss and Hentze 1999). The m⁷G also serves as a binding site for decapping enzymes that degrade the mRNA once it is no longer needed (Parker and Sheth 2007).

### Cap1

The 2′-O methylation in cap1 is used by a cell to differentiate its own (self) from viral RNA. Viral RNAs with 5′-ppp and double-stranded blunt ends, serve as a ligand for Retinoic Acid Inducible Gene-I (RIG-I)—a cytosolic innate immune receptor that can distinguish cellular self RNAs from pathogenic non-self RNAs. Once RIG-I is activated, it triggers a signaling pathway that leads to Type-I IFN production which ultimately destroys the viral RNA. It has recently been shown that cap0 double-stranded RNA activates RIG-I, but cap1 double-stranded RNA does not (Devarkar et al. 2016). Thus the 2′-O methylation of cap1 abrogates RIG-I activation. Many viruses have evolved a mechanism to cap their genomes and/or transcripts with cap1 (Reinisch, Nibert, and Harrison 2000) or snatch them from the host RNA (cap snatching) (Caton and Robertson 1980), both of which helps them evade the immune response by preventing recognition by RIG-I.

### Cap2

There is no consensus yet on the role of cap2 methylations. Cap2 methylations are reported to be present in as much as 50% of the transcripts (Whisenand et al. 2017). Furthermore, cap2 mRNA has been found to be 3-fold more enriched in polysomal fractions compared to non-polysomal fractions, whereas the amount of cap1 transcripts was the same in both fractions (Werner et al. 2011). This indicates that cap2-capped mRNA may have an increased affinity for ribosomes, or alternatively, methylation of cap2 occurs after the ribosomes bind to the mRNA. Recently, it has been found that methylations of second transcribed nucleotide in cap2 impacts protein production level in a cell-specific manner and contributes to RNA immune evasion (Drazkowska et al. 2022).

Cap m⁶Am

The N6 methylation in m⁷Gpppm⁶AmpN₂p-RNA transcripts increases the stability of the RNA against decapping when compared to m⁷GpppAmppN₂p-RNA transcripts (Pandey et al. 2020). The half-life of m⁷Gpppm⁶AmpN₂p-RNA transcripts is 2.5 hours higher than m⁷GpppN₁mpN₂p-RNA transcripts. Furthermore, the N6 methylation in m⁷Gpppm⁶AmpN₂p-RNA transcripts makes them less susceptible to microRNA-mediated degradation (Mauer et al. 2017).

TMG/m₃^{(2,2,7)}G/2,2,7-trimethylguanosine Cap

The TMG cap modifications are highly conserved in eukaryotes. TMG caps are believed to be necessary for the snRNAs to fulfil their cellular functions (Huber et al. 1998). TMG-capped ncRNAs have also been found to have higher expression levels compared to snRNAs lacking TMG caps (Jia et al. 2007). The terms m₃^{(2,2,7)}G and TMG are used interchangeably herein.

### Nad⁺ and NadH Caps

These caps have been found in bacteria and yeast (Cahová et al. 2015; Walters et al. 2017), and more recently in humans (Jiao et al. 2017) and plants as well (Y. Wang et al. 2019). NAD-capped transcripts constitute a small proportion of the total transcript pool from any gene, but they are enriched in the polysomal fraction and associate with the translating ribosomes (Y. Wang et al. 2019). In mitochondria, NAD⁺-capped RNA levels can reach up to 60% of mitochondrial transcripts (Bird et al. 2018). NAD gets incorporated in mRNA transcript by pol-II in a largely statistical manner that reflects the competition of NAD with the canonical initiator ATP (Zhang et al. 2020). Unlike canonical caps, which impart stability to their respective transcripts, NAD-caps have been shown to promote the decay of their respective transcripts (Jiao et al. 2017). Additionally, NAD-capped transcripts are, on average, shorter than non-NAD-capped transcripts, and are also not translatable in vitro (Zhang et al. 2020).

Whether NAD⁺-capped transcripts are capable of being translated is still somewhat unclear. The caps are present in mRNAs that are both spliced and poly-adenylated (Jiao et al. 2017; Walters et al. 2017), but these appear unable to be translated during in vitro translation experiments (Zhang et al. 2020). In contrast, polysome fractions from A. thaliana found an enrichment of NAD⁺-capped mRNAs associated with translating ribosomes (Y. Wang et al. 2019). Whether translation of NAD⁺-capped transcripts is particular to certain cells or species is therefore unknown, but it is possible that these transcripts could only be translated under specific circumstances and potentially make use of cap-independent translation through e.g., internal ribosome entry sites (IRES).

### FAD Caps

FAD caps appear to be enriched in shorter RNAs (<200 nt) (Doamekpor et al. 2020) and can be decapped (deFADed) by Nudt12 and Nudt16 (Sharma et al. 2020). The nature of RNAs capped with FAD caps is unknown because we currently do not have any method that can specifically enrich transcripts carrying these caps (Doamekpor et al. 2020).

### UDP-Glc and UDP-GlcNAc Caps

These uridine-containing NCINs compete with uridine triphosphate (UTP) for use by RNA polymerase as initiating nucleotides. The UDP-GlcNAc caps may be among the most abundant non-canonical caps, even more than NAD⁺, and have been shown to respond to oxidative and alkylation stresses in yeast (J. Wang et al. 2019). However, no enzymes involved in its processing have been discovered and no hypotheses as to its specific function have been forwarded.

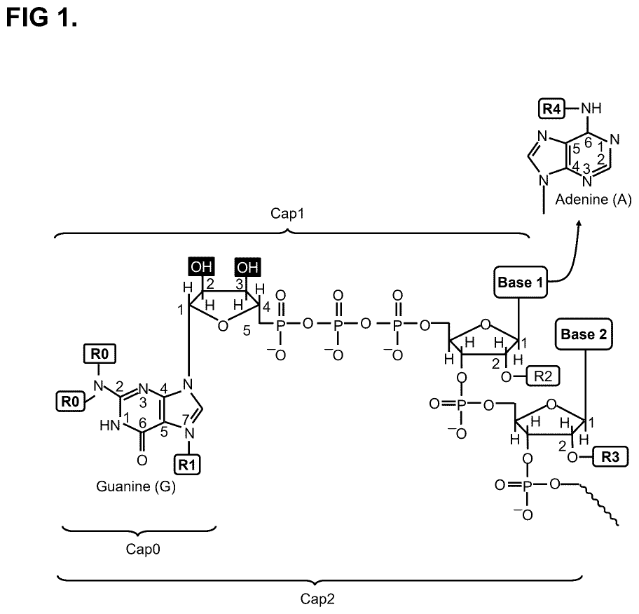

TABLE 1
Different canonical cap structures as depicted in
FIG. 1. N depicts any base, i.e. A, C, G or U.

| | | | | | | | Base | |
|---|---|---|---|---|---|---|---|---|
| | | Modifications and their loci | | | | | Base | Base |
| | | R0 | R1 | R2 | R3 | R4 | 1 | 2 |
| Cap | Cap0 | H | CH₃ | H | H | — | N | N |
| types | Cap1 | H | CH₃ | CH₃ | H | — | N | N |
| | Cap2 | H | CH₃ | CH₃ | CH₃ | — | N | N |
| | Cap2-1 | H | CH₃ | H | CH₃ | — | N | N |
| | Cap0 m⁶A | H | CH₃ | H | H | CH₃ | A | N |
| | Cap1 m⁶Am | H | CH₃ | CH₃ | H | CH₃ | A | N |
| | Cap2 m⁶Am | H | CH₃ | CH₃ | CH₃ | CH₃ | A | N |
| | Cap2-1 m⁶A | H | CH₃ | H | CH₃ | CH₃ | A | N |
| | TMG | CH₃ | CH₃ | H | H | — | N | N |
| | Unmethylated | H | H | H | H | — | N | N |

Examples of non-canonical caps are shown in FIG. 2.

The term “vicinal diol” relates to two diols bonded to adjacent carbons. This might be for example a 2′,3′-diol attached to the ribose sugar backbone of RNA. The diol may also be a 1′,2′-diol or a 3′,4′-diol. In FIGS. 1 and 2 vicinal diols are highlighted.

The term “polynucleotide adapter” means according to the present invention a synthetic oligonucleotide that extends the capped polynucleotide. The polynucleotide adapter may be RNA or DNA and may have different lengths. Also, the term an oligonucleotide extension (OTE) may be used. These terms are used herein interchangeably.

“Linker” may be a row of chemical groups between two polynucleotides. According to the present invention the polynucleotide adapter is ligated via a linker to the oxidized diol of the native 5′ cap. In some cases, the linker may be a bond or a chemical structure having amine groups at each end of the chain. The linker may be an ethylene diamine. The term “extended polynucleotide construct” according to the present invention refers to an RNA extended with a polynucleotide adapter via said linker.

If in the present invention a separate linker molecule in for ligating the polynucleotide adapter, i.e., oligonucleotide extension adapter (OTE), is used, the method of the invention may comprise the following steps:
A) The OTE is treated with sodium periodate to oxidize vicinal diols at the 3′ end. The product of the reaction is then reacted with ethylene diamine dichloride linker molecule in the presence of sodium cyanoborohydride. A reductive amination reaction takes place that joins the linker to the 3′ end of the OTE; a morpholine ring is created in the process.
B) The native capped RNA is treated with sodium periodate to oxidize vicinal diols at both the 5′ cap and 3′-most nucleotide.
C) Products of reactions in A and B are combined in the presence of sodium cyanoborohydride. This causes the previously prepared OTE to ligate with both the 5′ end and 3′ of the capped RNA. The OTE at the 3′ end is ligated in reverse orientation.
D) The reverse oriented OTE at the 3′-end of the capped RNA is removed by oligo(dT)-guided RNase H cleavage of the poly-A tail. Subsequent poly-A tailing is followed by the standard library preparation protocol for Nanopore sequencing.

If, on the other hand, the OTE possesses a pre-bound 3′ linker, i.e., the 3′ linker was already introduced during synthesis of the OTE, the invention may comprise the following steps:
A) The native capped RNA is first treated with sodium periodate to oxidize vicinal diols at both the 5′ cap and 3′-most nucleotide.
B) The oxidized capped RNA from step A is reacted with a synthetic oligonucleotide extension OTE with an aminated linker moiety at the 3′ end (OTE-linker-NH₂). The reaction is carried out in the presence of sodium cyanoborohydride. This causes the OTE to ligate with both the 5′ end and 3′ of the capped RNA. The OTE also gets ligated at the 3′ but in reverse orientation.
C) The reverse-oriented OTE at the 3′-end of the capped RNA is removed by oligo (dT)-guided RNase H cleavage of the poly-A tail. Subsequent poly-A tailing is followed by the standard library preparation protocol for Nanopore sequencing.

“At least a portion” means “at least partially” and relates to the sequencing of the extended polynucleotide construct. According to the present invention at least the 5′ native cap is sequenced.

“Sequencing” means direct sequencing of the extended polynucleotide construct, which according to the invention must comprise at least a portion of RNA, i.e. the capped RNA. Direct sequencing does not allow for sequencing information to be inferred through analysis of a related molecule, e.g. a cDNA. “Sequencing” may include base-calling some or all of the adapter sequence and/or some or all of the nucleotides of the capped RNA, optionally also some or all of any poly-A tail and/or ONT adaptor. “Sequencing” may include characterisation of the cap, in particular determination of the native cap structure. “Sequencing” may preferably include characterisation of the cap as well as base-calling. In addition, “sequencing” may include determination of the existence of sites of methylation, e.g. on N(A)₁ or N₂.

The term “nanopore sequencing” relates to feeding a molecule of interest through a pore or an opening (biological or solid-state) and characterizing the molecule of interest with the characteristic modulation of the current of voltage due to its translocation through the pore. Thus the nanopore sequencing may employ a solid-state nanopore system; solid state nanopores may be made, for example, of silicon nitride, graphene or other suitable materials. Biological pores are typically formed from proteins or nucleic acid, e.g. DNA.

“Sequencing motor protein” unwinds the double-stranded cDNA, or double stranded RNA-cDNA heteroduplex allowing single-stranded RNA/DNA in nanopore sequencing while a sensor measures ionic current.

“Illumina sequencing” relates to sequencing on an Illumina platform using their core “sequence by synthesis” technology as implemented in e.g., iSeq, MiSeq, MiniSeq, NextSeq, HiSeq, and NovaSeq.

The term “ionic current signature” refers to the characteristic perturbations in the pore current when a DNA, RNA or any other chemical structure passes through the constriction of the pore.

The term “periodate susceptible” means the capability of reaction with a periodate, e.g., sodium periodate. According to the present invention, caps that have a vicinal diol, e.g., 2′,3′ diol, are periodate susceptible.

The term “comprising” is meant not to be limiting to any subsequently stated elements but rather to encompass non-specified elements of major or minor functional importance. In other words, the listed steps, elements or options need not be exhaustive. Whenever the words “including” or “having” are used, these terms are meant to be equivalent to “comprising” as defined above. Moreover, the term “comprises” is meant to encompass the terms “consisting essentially of” and “consisting of”.

These and other aspects and embodiments of the inventions are disclosed and encompassed by the description and examples of the present invention. Further literature concerning any one of the materials, methods, uses and compounds to be employed in accordance with the present invention may be retrieved from public libraries and databases, using for example electronic devices. For example, the public database “Medline” or “PubMed” may be utilized, which is hosted by the National Center for Biotechnology Information and/or the National Library of Medicine at the National Institutes of Health. Further databases and web addresses, such as the virtual library “Martindale's center” are known to the person skilled in the art and can also be obtained using internet search engines.

The above disclosure generally describes the present invention. A more complete understanding can be obtained by reference to the following specific examples which are provided herein for purposes of illustration only and are not intended to limit the scope of the invention.

### EXAMPLES

### Example 1: 3′ Modification of Sequence Adapter (Oligonucleotide Extender (OTE)) with Diamine Linker

To form dialdehyde groups, from 2′,3′-cis-diols, on oligonucleotide extender (OTE) 3′ ends (see FIG. 4A), the following OTE oxidation reaction was assembled: 100 mM OTE and 8.3 mM sodium periodate (NaIO₄) in H₂O. The reaction was incubated for 15 min in room temperature (25° C.) in a fume hood. To stop the reaction, sodium hypophosphite (NaPO₂H₂) was added to a final concentration of 30 mM and further incubated for 20 min.

To attach diamine linker to oxidized OTE 3′ ends, the following reagents were added to the finished OTE oxidation reaction: 0.4 mM sodium acetate buffer (pH 4.5) and 5.8 mM ethylenediamine dihydrochloride (C₂H₁₀Cl₂N₂). This reaction was allowed to proceed for 15 min at 25° C. This reaction was completed by adding 3.3 mM sodium cyanoborohydride (NaBH₃CN) and by further incubation at 25° C. for 2 h.

To recover 3′ modified OTE (OTE-NH2) from the previous reaction, nucleotide material was precipitated with 2% lithium perchlorate in acetone at −20° C. for 2 h. Nucleotide material was then pelleted by centrifugation for 5 min at 10K g. RNA pellet was then dissolved in 50 ml 0.4M NaCl.

### Example 2: Oxidation of 2,3-cis-diols of m⁷G 5′ Cap Forming a 3′ and 5′ Dialdehyde

To form dialdehyde groups, from 2,3-cis-diols, on mRNA 5′ caps and 3′ terminal nucleotides (see FIG. 6A), the following mRNA oxidation reaction was assembled: 0.2 mM mRNA (From TriLink: CleanCap® EGFP mRNA-(L-7601)), 0.5 M Sodium acetate (pH 5.5) (NaAc) and 5 mM sodium periodate (NaIO₄) in H₂O. The reaction was incubated for 1 h in room temperature (25° C.) in a fume hood. To stop the reaction, sodium hypophosphite (NaPO₂H₂) was added to a final concentration of 0.2 M and further incubated for 10 min.

### Example 3: Ligation of OTE-NH₂ to 3′ and 5′ Dialdehyde Modified mRNA 5′-Cap

To ligate the OTE adapter, modified with an aminated 3′ linker molecular structure (OTE-NH₂) (see FIGS. 4A and 6B), to oxidized mRNA, the following reagents were added to the finished mRNA oxidation reaction: 0.4 mM sodium acetate buffer (pH 4.5) and 10 mM OTE-NH₂ (as prepared in Method of FIG. 4) or custom pre-modified OTE supplied by oligo synthesis service (Method of FIG. 6). This reaction was allowed to proceed for 15 min at 25° C. This reaction was completed by adding 3.3 mM sodium cyanoborohydride (NaBH₃CM) and by further incubation at 25° C. for 2 h. Note that OTE adapters may also ligate to mRNA 3′ ends in addition to 5′ caps (OTE-mRNA-OTE).

To recover the OTE-mRNA-OTE from the previous reaction, nucleotide material was precipitated with 2% lithium perchlorate in acetone at −20° C. for 2 h. Nucleotide material was then pelleted by centrifugation for 5 min at 10K g. The RNA pellet was then dissolved in 50 ml 0.4 M NaCl. RNA was further purified through size selection, to remove unligated OTE adapter, using 0.7× volume of AMPure XP Reagent (Beckman Coulter), following recommended protocol from manufacturer.

### Example 4: Removal of 3′ mRNA OTE Adapters Using RNase H

To remove the OTE adapters attached to OTE-mRNA-OTE 3′ ends (FIG. 6B, C), mRNA poly A tails were targeted for RNA degradation using RNase H. This reaction was achieved by first mixing 0.2 mM OTE-mRNA-OTE with 10 mM Oligo dT (15) primers. To form double stranded Poly A RNA: DNA duplex, the sample was heated to 80° C. before cooling slowly (10 minutes) to 37° C. Double stranded RNA: DNA duplexes were then digested in 1× RNase H buffer by adding 0.25 Units RNase H (New England Biolabs) per pmol OTE-mRNA-OTE. The reaction was carried out for 20 minutes at 37° C. RNase H was then inactivated by adding 5 mM EDTA. OTE-mRNA PolyA was then purified using 1× volume of AMPure XP Reagent (Beckman Coulter), following the recommended protocol from the manufacturer.

### Example 5: Sequencing of Extended RNA Construct (OTE-mRNA) Using Oxford Nanopore Technology (ONT)

To allow sequencing using ONT, OTE-mRNA PolyA was re-polyadenylated using Poly A polymerase (New England Biolabs), following the manufacturers recommended protocol. The poly A reaction was carried out for 15 min at 37° C. before inactivation of Poly A polymerase at 75° C. for 5 minutes.

OTE-mRNA was then prepared for direct RNA sequencing using the RNA002 kit from Oxford Nanopore, following the recommended protocol from manufacturer; see FIG. 3. The sample was then sequenced with a MinION sequencing device on a Flongle flow cell. Readthrough was successfully obtained across the m⁷G cap and NAD caps (OTE-mRNA junction) as shown in FIGS. 5A and 5B and for m⁷G cap as shown in FIG. 7.

### Example 6: Analysis

To identify the different caps in an RNA sample-of-interest, first a machine learning classifier needs to be trained on the current signature for different caps. Towards this end, synthetic RNA sequences with known caps are adapted and then sequenced on the Nanopore. The Nanopore current-based features corresponding to the cap and the cap-adjacent bases are then used to train the machine learning classifier.

For classification of caps in an RNA sample-of-interest, the nanopore current-based features corresponding to the cap and the cap-adjacent bases in adapted reads are fed into the pre-trained classifier, which identifies the cap type on each individual RNA molecule. The cap-adjacent RNA sequence is used to identify the RNA transcript/gene. In this way, both the RNA sequence and its respective cap type are characterized. This yields a profile/map with a quantification of caps and their respective RNA transcript sequences in the sample.

For discovery of disease-related genetic markers or genetic markers relating to specific healthy conditions, the profile/map of caps and their respective transcripts are compared across disease and healthy samples or across subject and control samples to identify differentially capped RNA transcripts. These transcripts are then subjected to further investigation.

### Example 7: Identification of 5′-Cap Disease Markers Using Cap Sequencing

A medicinal evaluation using cap sequencing is carried out with the following steps:
1. Sample collection (tissue biopsies, blood), 2. RNA extraction, 3. mRNA extraction 4. Cap sequencing (as described in Examples 1-5). 5. Analysis of relevant genetic markers (as described in Example 6).

SEQUENCE LISTING
<210> 1
<211> 100
<212> RNA
<213> Artificial
<220>
<223> Polynucleotide adapter
<400> 1
ggagagagua gcaacgcgaa ggaugaggaa gcggaagaag
60
agucaacagg aaccacaaga ggacucaugu agcugaucga
100
ugcgacuagc uacquacugu
<210> 2
<211> 59
<212> RNA
<213> artificial sequence
<220>
<223> Polynucleotide adapter
<400> 2
gagaugagcu uucguucguc uccggacuua ucgcaccacc
59
uauccaucau caguacugu

### REFERENCES

Bird, Jeremy G., Urmimala Basu, David Kuster, Aparna Ramachandran, Ewa Grudzien-Nogalska, Atif Towheed, Douglas C. Wallace, et al. 2018. “Highly Efficient 5′ Capping of Mitochondrial RNA with NAD+ and NADH by Yeast and Human Mitochondrial RNA Polymerase.” ELife 7 (December). https://doi.org/10.7554/eLife.42179.
Bjornsti, Mary-Ann, and Peter J. Houghton. 2004. “Lost in Translation: Dysregulation of Cap-Dependent Translation and Cancer.” Cancer Cell 5 (6): 519-23.
Cahová, Hana, Marie-Luise Winz, Katharina Höfer, Gabriele Nübel, and Andres Jäschke. 2015. “NAD CaptureSeq Indicates NAD as a Bacterial Cap for a Subset of Regulatory RNAs.” Nature 519 (7543): 374-77.
Caton, A. J., and J. S. Robertson. 1980. “Structure of the Host-Derived Sequences Present at the 5′ Ends of Influenza Virus MRNA.” Nucleic Acids Research 8 (12): 2591-2603.
Cowling, Victoria H. 2019. “CAPAM: The MRNA Cap Adenosine N6-Methyltransferase.” Trends in Biochemical Sciences 44 (3): 183-85.
Devarkar, Swapnil C., Chen Wang, Matthew T. Miller, Anand Ramanathan, Fuguo Jiang, Abdul G. Khan, Smita S. Patel, and Joseph Marcotrigiano. 2016. “Structural Basis for M7G Recognition and 2′-O-Methyl Discrimination in Capped RNAs by the Innate Immune Receptor RIG-I.” Proceedings of the National Academy of Sciences of the United States of America 113 (3): 596-601.
Devarkar, Wang, and Miller. n.d. “Structural Basis for m⁷G Recognition and 2′-O-Methyl Discrimination in Capped RNAs by the Innate Immune Receptor RIG-I.” Proceedings of the Estonian Academy of Sciences. Biology, Ecology=Eesti Teaduste Akadeemia Toimetised. Bioloogia, Okoloogia. https://www.pnas.org/content/113/3/596.short.
Doamekpor, Selom K., Ewa Grudzien-Nogalska, Agnieszka Mlynarska-Cieslak, Joanna Kowalska, Megerditch Kiledjian, and Liang Tong. 2020. “DXO/Rai1 Enzymes Remove 5′-End FAD and Dephospho-CoA Caps on RNAs.” Nucleic Acids Research 48 (11): 6136-48.
Drazkowska, Karolina, Natalia Baran, Marcin Warminski, Rafal Tomecki, Anaïs Depaix, Dominik Cysewski, Renata Kasprzyk, Joanna Kowalska, Jacek Jemielity, and Pawel J. Sikorski. 2022. “2′-O-Methylation of the Second Transcribed Nucleotide within MRNA 5′ Cap Impacts Protein Production Level in a Cell Specific Manner and Contributes to RNA Immune Evasion.” BioRxiv. https://doi.org/10.1101/2022.02.03.478939.
Dunn, Sianadh, Olivia Lombardi, Radoslaw Lukoszek, and Victoria H. Cowling. 2019. “Oncogenic PIK3CA Mutations Increase Dependency on the mRNA Cap Methyltransferase, RNMT, in Breast Cancer Cells.” Open Biology 9 (4): 190052.
Efimov, V. A., O. G. Chakhmakhcheva, J. Archdeacon, J. M. Fernandez, O. N. Fedorkin, Yu L. Dorokhov, and J. G. Atabekov. 2001. “Detection of the 5′-Cap Structure of Messenger RNAs with the Use of the Cap-Jumping Approach.” Nucleic Acids Research 29 (22): 4751-59.
Hu, Hao, Nora Flynn, and Xuemei Chen. 2021. “Discovery, Processing, and Potential Role of Noncanonical Caps in RNA.” In Epitranscriptomics, edited by Stefan Jurga and Jan Barciszewski, 435-69. Cham: Springer International Publishing.
Huber, J., U. Cronshagen, M. Kadokura, C. Marshallsay, T. Wada, M. Sekine, and R. Lührmann. 1998. “Snurportin1, an M3G-Cap-Specific Nuclear Import Receptor with a Novel Domain Structure.” The EMBO Journal 17 (14): 4114-26.
Hudeček, Oldřich, Roberto Benoni, Paul E. Reyes-Gutierrez, Martin Culka, Hana Šanderová, Martin Hubálek, Lubomír Rulíšek, Josef Cvačka, Libor Krásný, and Hana Cahová. 2020. “Dinucleoside Polyphosphates Act as 5′-RNA Caps in Bacteria.” Nature Communications 11 (1): 1052.
Jia, Dong, Lun Cai, Housheng He, Geir Skogerbø, Tiantian Li, Muhammad Nauman Aftab, and Runsheng Chen. 2007. “Systematic Identification of Non-Coding RNA 2,2,7-Trimethylguanosine Cap Structures in Caenorhabditis Elegans.” BMC Molecular Biology 8 (September): 86.
Jiao, Xinfu, Selom K. Doamekpor, Jeremy G. Bird, Bryce E. Nickels, Liang Tong, Ronald P. Hart, and Megerditch Kiledjian. 2017. “5′ End Nicotinamide Adenine Dinucleotide Cap in Human Cells Promotes RNA Decay through DXO-Mediated DeNADding.” Cell 168 (6): 1015-1027.e10.
Lewis, J. D., and E. Izaurralde. 1997. “The Role of the Cap Structure in RNA Processing and Nuclear Export.” European Journal of Biochemistry/FEBS 247 (2): 461-69.
Mauer, Jan, Xiaobing Luo, Alexandre Blanjoie, Xinfu Jiao, Anya V. Grozhik, Deepak P. Patil, Bastian Linder, et al. 2017. “Reversible Methylation of M6Am in the 5′ Cap Controls MRNA Stability.” Nature 541 (7637): 371-75.
Merenkova, I. N., and Jbdm Edwards. 2000. Method for the specific coupling of the cap of the 5′end of an mRNA fragment and preparation of mRNA and complete cDNA. U.S. Pat. No. 6,022,715, issued 2000. https://patents.google.com/patent/U.S. Pat. No. 6,022,715A/en.
Mulroney, Logan, Mark Akeson, Miten Jain, and Hugh Olsen. 2021. Methods of Analyzing Capped Ribonucleic Acids. USPTO 20210348224: A1. US Patent, filed May 22, 2019, and issued Nov. 11, 2021. https://patentimages.storage.googleapis.com/15/65/2f/e66cc72d86ad26/US20210 348224A1.pdf.
Pandey, Radha Raman, Elena Delfino, David Homolka, Adriana Roithova, Kuan-Ming Chen, Lingyun Li, Giulia Franco, et al. 2020. “The Mammalian Cap-Specific M6Am RNA Methyltransferase PCIF1 Regulates Transcript Levels in Mouse Tissues.” Cell Reports 32 (7): 108038.
Parker, Roy, and Ujwal Sheth. 2007. “P Bodies and the Control of MRNA Translation and Degradation.” Molecular Cell 25 (5): 635-46.
Preiss, T., and M. W. Hentze. 1999. “From Factors to Mechanisms: Translation and Translational Control in Eukaryotes.” Current Opinion in Genetics & Development 9 (5): 515-21.
Reinisch, K. M., M. L. Nibert, and S. C. Harrison. 2000. “Structure of the Reovirus Core at 3.6A Resolution.” Nature 404 (6781): 960-67.
Schuberth-Wagner, Christine, Janos Ludwig, Ann Kristin Bruder, Anna-Maria Herzner, Thomas Zillinger, Marion Goldeck, Tobias Schmidt, et al. 2015. “A Conserved Histidine in the RNA Sensor RIG-I Controls Immune Tolerance to N1-2′O-Methylated Self RNA.” Immunity 43 (1): 41-51.
Sharma, Sunny, Ewa Grudzien-Nogalska, Keith Hamilton, Xinfu Jiao, Jun Yang, Liang Tong, and Megerditch Kiledjian. 2020. “Mammalian Nudix Proteins Cleave Nucleotide Metabolite Caps on RNAs.” Nucleic Acids Research 48 (12): 6788-98.
Walters, Robert W., Tyler Matheny, Laura S. Mizoue, Bhalchandra S. Rao, Denise Muhlrad, and Roy Parker. 2017. “Identification of NAD+ Capped MRNAs in Saccharomyces Cerevisiae.” Proceedings of the National Academy of Sciences of the United States of America 114 (3): 480-85.
Wang, Jin, Bing Liang Alvin Chew, Yong Lai, Hongping Dong, Luang Xu, Seetharamsingh Balamkundu, Weiling Maggie Cai, et al. 2019. “Quantifying the RNA Cap Epitranscriptome Reveals Novel Caps in Cellular and Viral RNA.” Nucleic Acids Research 47 (20): e130-e130.
Wang, Yuan, Shaofang Li, Yonghui Zhao, Chenjiang You, Brandon Le, Zhizhong Gong, Beixin Mo, Yiji Xia, and Xuemei Chen. 2019. “NAD+-Capped RNAs Are Widespread in the Arabidopsis Transcriptome and Can Probably Be Translated.” Proceedings of the National Academy of Sciences of the United States of America 116 (24): 12094-102.
Werner, Maria, Elżbieta Purta, Katarzyna H. Kaminska, Iwona A. Cymerman, David A. Campbell, Bidyottam Mittra, Jesse R. Zamudio, Nancy R. Sturm, Jacek Jaworski, and Janusz M. Bujnicki. 2011. “2′-O-Ribose Methylation of Cap2 in Human: Function and Evolution in a Horizontally Mobile Family.” Nucleic Acids Research 39 (11): 4756-68.
Whisenand, Josh M., Krist T. Azizian, Jordana M. Henderson, Sabrina Shore, Dongwon Shin, Alexandre Lebedev, Anton P. McCaffrey, and Richard I. Hogrefe. 2017. “Considerations for the Design and CGMP Manufacturing of MRNA Therapeutics.” San Diego, CA: TriLink BioTechnologies. https://www.trilinkbiotech.com/media/contentmanager/content/mRNA_OTS1.pdf.
Wulf, Madalee G., John Buswell, Siu-Hong Chan, Nan Dai, Katherine Marks, Evan R. Martin, George Tzertzinis, Joseph M. Whipple, Ivan R. Corrêa Jr, and Ira Schildkraut. 2019. “The Yeast Scavenger Decapping Enzyme DcpS and Its Application for in Vitro RNA Recapping.” Scientific Reports 9 (1): 8594.
Zhang, Yaqing, David Kuster, Tobias Schmidt, Daniel Kirrmaier, Gabriele Nübel, David Ibberson, Vladimir Benes, Hans Hombauer, Michael Knop, and Andres Jäschke. 2020. “Extensive 5′-Surveillance Guards against Non-Canonical NAD-Caps of Nuclear MRNAs in Yeast.” Nature Communications. https://doi.org/10.1038/s41467-020-19326-3.

1. A method of characterizing a capped ribonucleic acid (RNA) using sequencing, wherein the capped RNA is a ribonucleic acid (RNA) with its native 5′ cap, the method comprising the steps of:
i) oxidation of the vicinal diol of the native 5′ cap of the capped RNA;
ii) ligation of a polynucleotide adapter via a linker to the oxidized diol of the native 5′ cap providing an extended polynucleotide construct and
iii) sequencing at least a portion of the extended polynucleotide construct, wherein said portion includes the native cap.

2. The method of claim 1, wherein the method comprises a further step of reductive amination of the oxidized vicinal diol formed in i) between steps i) and ii).

3. The method of claim 1 or 2, wherein the method comprises the following further steps between steps ii) and iii): nucleotide material precipitation, RNA purification, poly-A removal and/or poly-A tailing.

4. The method of any one of the preceding claims, wherein the sequencing is carried out with nanopore sequencing.

5. The method of any one of the preceding claims, wherein the linker comprises amine groups.

6. The method of any one of the preceding claims, wherein the polynucleotide adapter comprises an introduced amino group.

7. The method of any one of the preceding claims, wherein sequencing information generated in iii) is inputted into a classifier of sequence information in order to characterise said capped RNA.

8. The method of claim 7 wherein sequencing information generated in iii) is inputted into a classifier of sequence information in order to identify the native 5′ cap of said capped RNA.

9. The method of any one of the preceding claims, wherein the vicinal diol is a 2′,3′, 1′,2′ or 3′,4′ diol.

10. The method of any one of the preceding claims, wherein the polynucleotide adapter may be ligated to the 3′ end of the RNA in addition to the 5′ end.

11. The method of any one of the preceding claims, wherein a sequencing motor protein attached to the polynucleotide adapter is ligated to the 5′ end of the RNA.

12. The method of any one of claims 4 to 11, wherein nanopore sequencing occurs in the 3′ to the 5′ direction.

13. The method of any one of claims 4 to 12, wherein the method comprises characterizing the extended RNA construct by ionic current signature produced during its translocation through the nanopore.

14. The method of any one of the preceding claims, wherein a plurality of native 5′ caps present in a sample of polynucleotide constructs may be sequenced using a single assay.

15. The method of any one of the preceding claims, wherein the native 5′ cap contains a periodate-susceptible vicinal diol.

16. The method of any one of the preceding claims, wherein the native 5′ cap is selected from the group consisting of tri-methylated m⁷G, m⁷G, G, NADH, NAD⁺, FAD, Glc-UDP, GlcNAc-UDP and NpₙN.

17. The method of any one of the preceding claims, wherein the polynucleotide adapter is RNA or DNA.

18. A method of identifying whether a genetic marker specific for a condition is present in a sample wherein the method comprises the steps:
i) obtaining an mRNA sample of a subject
ii) carrying out the method of any one of claims 1 to 17
iii) analyzing one or more genetic markers specific for the condition
iv) comparing the sequence of the mRNA sample with a control sample and
v) evaluating the results.

19. The method of claim 18, wherein the condition is a cancer, viral disease, bacterial disease or an autoimmune disease.

20. Kit for characterizing a ribonucleic acid with its native 5′ cap comprising the reagents for the method as defined in any one of claims 1 to 19 and instructions for carrying out the method.

21. A non-transitory computer readable medium comprising instructions for the method of characterizing the native 5′ capped RNA in claims 1 to 19

22. A computing device comprising a processor and the non-transitory computer readable medium of claim 21.

23. The computing device of claim 22, wherein the computing device is part of a system comprising a nanopore sequencing device.

24. A method of characterising an RNA with a native 5′ cap, which method comprises sequencing at least a portion of a polynucleotide construct comprising said capped RNA and a polynucleotide adapter ligated via a linker to said cap, wherein the linking moiety is formed from the vicinal diol of the native 5′ cap, and wherein said portion includes the native 5′ cap.

25. The method of claim 24 wherein the polynucleotide adapter is ligated via a linker to said cap through a morpholine ring.

26. The method of claim 24 or claim 25 wherein the sequencing, linker, adapter or cap are as claimed in any preceding claim.

27. A method of any one of claims 1 to 17, wherein said sequencing step (iii) includes determination of the native cap structure.


SEQUENCE LISTING (2 sequences)

SEQ ID NO: 1 - RNA, 100 nt, synthetic construct
  source 1..100: mol_type = other RNA
ggagagagtagcaacgcgaaggatgaggaagcggaagaagagtcaacaggaaccacaagaggactcatgtagctgatcgatgcgactagctacgtactgt

SEQ ID NO: 2 - RNA, 59 nt, synthetic construct
  source 1..59: mol_type = other RNA
gagatgagctttcgttcgtctccggacttatcgcaccacctatccatcatcagtactgt